(19) United States
(12) Patent Application Publication
(10) Pub. No.: US 2010/0272651 A1
(43) Pub. Date: Oct. 28, 2010

(54) METHOD FOR ASSESSING POTENTIAL FOR TUMOR DEVELOPMENT AND METASTASIS

(75) Inventors: Irene Georgakoudi, Acton, MA (US); Derrick Hwu, Carlisle, MA (US); Steven Boutrus, Salem, NH (US); Charlotte Kuperwasser, Boston, MA (US)

Correspondence Address: NUTTER MCCLENNEN & FISH LLP, SEAPORT WEST, 155 SEAPORT BOULEVARD, BOSTON, MA, 02210-2604, US

(73) Assignee: TUFTS UNIVERSITY, Boston, MA (US)

(21) Appl. No.: 12/764,566

(22) Filed: Apr. 21, 2010

Related U.S. Application Data
(60) Provisional application No. 61/171,634, filed on Apr. 22, 2009.

(51) Int. Cl. A61K 49/00; C12Q 1/06

(52) U.S. Cl. 424/9.6; 435/38

(57) ABSTRACT

The present invention generally provides methods for assessing the potential of tumor formation and/or metastasis using a combination (e.g., a ratio) of the number of circulating tumor cells and the number of circulating cells exhibiting autofluorescence within a selected wavelength region (e.g., red autofluorescence). In one aspect, it is directed to a method for providing likelihood of occurrence of a primary and/or a metastatic cancerous tumor in an animal, which comprises inoculating the animal with a plurality of cancer cells, determining a ratio of a number of cancer cells relative to a number of circulating indicator cells (e.g., immature leukocytes) that exhibit autofluorescence in the inoculated animal's blood and correlating the ratio to a likelihood that the animal will develop at least one primary and/or metastatic cancerous tumor, e.g., by way of assigning a probability for tumor development and/or metastasis based on the measured ratio. The method can also be utilized in human studies using, e.g., contrast agents to identify the circulating tumor cells.

### RELATED APPLICATION

The present application claims priority to a provisional application entitled “Method For Assessing Potential For Tumor Development And Metastasis” filed on Apr. 22, 2009 having a Ser. No. 61/171,634. This provisional application is herein incorporated by reference in its entirety.

### BACKGROUND OF THE INVENTION

The present invention relates generally to methods and apparatus for assessing progression of cancer, and more particularly, to such methods that can be employed to predict the likelihood of formation and/or metastasis of tumors.

In the United States, a large number of cancer cases develop each year, many of which result in death. The four major types of cancer are prostate, breast, lung, and colon cancer that account for approximately 50% of the new cancer cases and deaths. The majority of these deaths are due to cancer metastasis rather than primary cancerous tumors. In fact, primary cancerous tumors can often be eliminated through surgical and radiochemical treatments. Secondary tumors that develop due to metastasis are, however, more difficult to diagnose and treat.

While significant advances have been made in the understanding of primary and metastatic tumor development especially in the context of genetics and stromal-epithelial cell interactions, the present methods for assessing cancer progression suffer from a number of shortcomings. For example, the predictive ability of such methods can be limited. Furthermore, many methods can require extensive processing steps and, hence, can be difficult to implement. The shortcomings of present methods can often lead to inefficiencies in experimental animal cancer studies. For example, implantation of cancer cells in an animal does not necessarily lead to tumor formation. Generally, researchers need to wait 8-12 weeks following such implantation to determine whether an animal has developed tumor(s) so that it can be used in an experimental cancer study. Moreover, the shortcomings of the present methods for assessing metastatic potential of tumors in humans can lead to sub-optimal treatment protocols.

Accordingly, there is a need for enhanced methods and apparatus for assessing tumor formation and metastasis.

### SUMMARY OF THE INVENTION

The present invention generally provides methods and apparatus for assessing the potential of tumor formation and/or metastasis. In many embodiments, the likelihood of tumor formation and/or metastasis is determined based on a function of a number of circulating cancer cells and a number of circulating indicator cells, e.g., a ratio of the number of circulating cancer cells relative to the circulating indicator cells. By way of example, the number of each cell type can correspond to a volume density of the cells or the number of cells of each type determined within the same volume portion (e.g., the number of cells counted via in-vivo flow cytometry within a focal volume of radiation incident on circulating blood).

In one aspect, the invention is directed to a method for providing the likelihood of occurrence of a cancerous tumor in an animal, which comprises inoculating the animal with a plurality of cancer cells, determining a ratio of a number of cancer cells relative to a number of circulating non-cancer cells that exhibit autofluorescence in the inoculated animal's blood in a range of about 650 nm to about 690 nm in response to excitation with a wavelength of about 633 nm, and correlating the ratio to a likelihood that the animal will develop at least one cancerous tumor, e.g., by way of assigning a probability for tumor development based on the measured ratio. The circulating non-cancer cells that exhibit autofluorescence in the above wavelength range can be cells that exhibit markers for immaturity. For example, these non-cancer circulating cells can be immature leukocytes. The likelihood of tumor formation increases as the ratio increases. By way of example, in some cases, a probability greater than about 0.85 can be assigned for tumor formation if the ratio is greater than about 0.26. Throughout this application, the circulating non-cancer cells that exhibit autofluorescence in the above wavelength range (which can be immature leukocytes) are referred to as indicator cells. In some cases, the cancer cells and the indicator cells can be counted, e.g., concurrently or separately during similar time intervals (e.g., 0 hours to about 14 days or greater than 14 days after inoculating the animal), in-vivo in the animal's circulating blood, e.g., by employing methods of in-vivo flow cytometry, to derive the ratio (e.g., by dividing the respective counts). In other cases, the volume densities of the cancer cells and/or the indicator cells can be determined ex-vivo and utilized to obtain the ratio.

In another aspect, a method for providing the likelihood of occurrence of a cancerous tumor in an animal is disclosed that comprises inoculating the animal with a plurality of cancer cells that express a fluorescent protein (e.g., GFP), counting in-vivo the cancer cells in the animal's circulating blood by exciting the fluorescent protein and detecting fluorescent radiation emitted by the protein in response to the excitation, and counting in-vivo circulating cells that emit autofluorescent radiation at a wavelength in a range of about 650 nm to about 690 nm in response to excitation radiation with a wavelength of about 633 nm. A ratio of the count of the cancer cells relative to the count of the indicator cells is calculated and a likelihood is determined based on the ratio that at least one cancerous tumor will develop in the animal. In some cases, a probability can be assigned for tumor formation based on a particular numerical range in which the ratio resides.

In another aspect, the invention provides a method for determining metastatic potential of a tumor, which comprises determining a ratio of a number of cancer cells to a number of circulating indicator cells in a subject's blood (e.g., in blood circulating through the subject's vasculature or a volume of blood extracted from the subject) and correlating the ratio to a likelihood for metastasis of the tumor such that the lower the ratio the less likely for the tumor to metastasize.

In a related aspect, in the above method a predictive ratio greater than about 0.26 can indicate a likelihood of greater than about 85% that a tumor might be formed and a 50% likelihood of metastasis, while a ratio greater than about 0.5 can indicate a likelihood of about 100% that an existing tumor might metastasize.

In some cases, the ratio can be determined by counting the cancer cells and/or circulating indicator cells in-vivo in the patient's circulating blood. In other cases, the ratio can be determined by counting the cancer cells and/or the indicator cells ex-vivo, e.g., in a blood sample extracted from the subject. The ratio can be determined during a time interval in a range of 0 hours to about 14 days or can be determined greater than 14 days after inoculating the animal.

Another aspect of the invention can include a method for providing a likelihood of occurrence of a cancerous tumor in a patient, which comprises counting in-vivo cancer cells in the patient's circulating blood that emit autofluorescent radiation at a wavelength less than about 605 nm in response to excitation radiation at a wavelength of about 488 nm, counting in-vivo circulating cells that emit autofluorescent radiation at a wavelength in a range of about 650 nm to about 690 nm in response to excitation radiation with a wavelength of about 633 nm, calculating a ratio of the count of the cancer cells relative to the count of the circulating cells that exhibit autofluorescence in the range of about 650 nm to about 690 nm and determining a likelihood based on the ratio that at least one metastatic tumor will develop in the patient. The counting of the cancer cells and the circulating cells that exhibit autofluorescence in the range of about 650 nm to about 690 nm can be performed during similar, and preferably identical, time intervals such that the ratio of the counts would be indicative of the ratio of the volume densities of those cells. The circulating cancer cells could also be identified using: a) unique light scattering signatures or b) in-vivo fluorescent tagging of specific cancer cell antigens or receptors. As the ratio increases, the likelihood of metastasis also increases.

In many embodiments of the above methods, once the likelihood of tumor formation and/or metastasis is determined, certain actions can be taken based on the likelihood. For example, in animal studies, those inoculated animals that exhibit a high probability of tumor formation (e.g., a probability greater than about 50% or preferably greater that about 60% or 70%) can be retained and others can be discarded. In some other cases, once the likelihood of metastasis of a human tumor is determined, a therapy regimen can be devised, e.g., more aggressive treatments can be applied for tumors that exhibit a higher likelihood of metastasis, for example, a likelihood greater than about 50% or greater than about 60%.

Further understanding of various aspects of the invention can be obtained by reference to the following detailed description in conjunction with the associated drawings, which are described briefly below.

### BRIEF DESCRIPTION OF THE DRAWINGS

FIG. 1 is a flow chart depicting various steps in an embodiment of a method of the invention;

FIG. 2 is a flow chart depicting various steps in an exemplary embodiment of a method according to the teachings of the invention for assessing tumor formation in animals inoculated with cancer cells;

FIG. 3 is a flow chart depicting various steps in an exemplary embodiment of a method according to the teachings of the invention for assessing tumor metastasis in patients;

FIG. 4 is a schematic depiction of a two-color in-vivo flow cytometry system according to an exemplary embodiment of the invention;

FIG. 5 shows representative fluorescence peaks of green fluorescence protein (GFP) expressing human breast cancer cells implanted in mice, which were detected by in-vivo flow cytometry;

FIG. 6 shows representative red autofluorescence peaks emitted by circulating cells of the mice in which GFP expressing breast cancer cells were implanted;

FIG. 7 shows the average number of green fluorescent cells detected for different groups of mice including a control group and mice implanted with GFP expressing breast cancer cells;

FIG. 8 shows the average ratio of green fluorescence emitting cells to red autofluorescence emitting cells for each group of mice of FIG. 7;

FIG. 9 shows the total number of peaks detected within the first seven days post implantation in the green fluorescence channel as a function of the ratio of total green fluorescence peaks to total red fluorescence peaks detected also during the first seven days post implantation;

FIG. 10 shows overlap of FITC peaks with red autofluorescent peaks from mice blood cells labeled with FITC-CD31 antibodies; and

FIG. 11 shows overlap of FITC peaks with red autofluorescent peaks from mice blood cells labeled with FITC-Sca-1 antibodies.

### DETAILED DESCRIPTION OF THE INVENTION

The invention generally provides methods and apparatus for assessing the formation of primary tumor(s) and/or metastasis of existing tumor(s). By way of example, in some embodiments, the ratio of circulating cancer cells (e.g., cancer cells in the circulating blood) relative to circulating non-cancer cells that exhibit autofluorescence in a range of about 650 nm to about 690 nm in response to excitation at a wavelength of about 633 nm (which can be immature cells such as immature leukocytes) is measured and a likelihood of tumor formation and/or metastasis is determined based on that ratio. In general, as the ratio increases so does the likelihood of tumor formation and/or metastasis. The ability to determine the likelihood of tumor formation and/or metastasis can provide a number of advantages, e.g., it can allow early intervention in treating human cancers and increased efficiency in conducting experimental cancer studies based on animal models.

The terms used herein adhere to standard definitions generally accepted by those having ordinary skill in the art. In case any further explanation might be needed, some terms have been further elucidated below.

The term “cancerous” as used herein is intended to refer to any abnormal cells that divide without control characterized by the proliferation of anaplastic cells that can invade surrounding tissues and metastasize to new body sites.

The terms “metastasis,” “metastatic” and “metastasize” as used herein are intended to refer to the spread of malignant cells from one part of the body to another or the movement of cancerous cells through the basement membrane.

The terms “autofluorescence” and “autofluorescent” as used herein are intended to refer to the intrinsic fluorescence of cells that contain molecules which become fluorescent when excited by radiation of suitable wavelengths.

The terms “circulating cell” refers to any cell type found within the circulatory system of a subject and can comprise, but is not limited to, leukocytes, endothelial cells, neuronal cells, vascular cells, myocytes and mesenchymal cells, or any cell type found within the circulation of the subject. The term “non-cancer cell” refers to a cell that has not been previously identified as cancerous or abnormal.

The term “leukocyte” as used herein to refer to any white blood cell. The term leukocyte comprises, but is not limited to, hematopoietic stem cells, hematopoietic progenitor cell, granulocytes, macrophages, megakaryocytes, myelocytes, myeloblasts, B-cells, T-cells, monocytes, basophils, neutrophils, eosinophils, natural killer cells and any precursor thereof.

The term “immature leukocyte” as used herein refers to a non-terminally differentiated hematopoietic cell, such as, but not limited to, hematopoietic stem cells, hematopoietic progenitor cells, blast cells and any precursor cell thereof.

The term “subject” refers to any living organism. The term subject comprises, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered. In preferred embodiments, the subject is a mammal, including humans and non-human mammals. In the most preferred embodiment, the subject is a human.

The terms “sample,” “sample from a subject” and “extracted sample” as used herein refer to a small quantity of fluid of a subject, which can be obtained, e.g., by employing methods known in the art. Such a fluid, e.g., blood, can contain cancer cells, leukocytes or both. The term sample comprises, but is not limited to, blood, lymph fluid, sputum, saliva, spinal fluid, semen and any other bodily fluid or secretion of a subject.

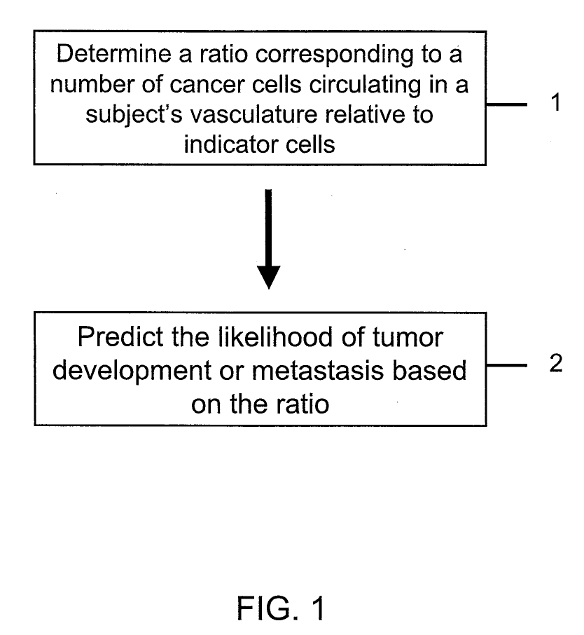

As noted above, in one aspect, the invention generally relates to a methodology for predicting tumor formation and/or metastasis in a subject. With reference to the flow chart of FIG. 1, an exemplary embodiment of a method for predicting primary or metastatic tumor formation in a subject (animal or human) can include determining a ratio (herein also referenced to as “predictive ratio”) corresponding to the number of cancer cells circulating in the subject's vasculature relative to circulating indicator cells (e.g., circulating immature leukocytes) (step 1). A likelihood for the formation of a tumor and/or the metastasis of an existing tumor can then be determined based on the ratio (step 2). In many cases, the ratio can be expressed as the ratio of volume density of circulating cancer cells to that of circulating indicator cells. For example, as discussed in more detail below, the ratio can be determined by measuring a volume density of cancer cells and that of indicator cells in a subject's blood, e.g., by employing in-vivo techniques to measure the volume densities in a sample volume, and dividing the two volume densities. In general, as the ratio increases, the probability of tumor formation or its metastasis also increases. For example, in some cases, a ratio greater than about 0.26 can indicate a likelihood of greater than about 85% percent that a tumor might be formed and a 50% likelihood of metastasis, while a ratio greater than about 0.5 can indicate a likelihood of about 100% that an existing tumor might metastasize. Likewise when the ratio decreases, the probability of tumor formation or its metastasis also decreases.

The predictive ratio of the circulating cancer cells to circulating indicator cells can be determined in a variety of ways. In some embodiments, the predictive ratio can be determined by employing in-vivo flow cytometry as the cells circulate through a live subject. For example, two-color in-vivo flow cytometry (IVFC) can be employed to excite the circulating cells at one wavelength for detecting cancer cells and at another wavelength for detecting the circulating indicator cells.

By way of example, cancer cells can be fluorescently labeled and detected as they move through a live subject. Examples of such methods for detection of cancer cells can be found in an article entitled “In Vivo Flow Cytometry” by Georgakoudi et al. and published in Cancer Research, vol. 64, pg. 5044 (2004), and an article entitled “Portable two-color in vivo flow cytometer for real-time detection of fluorescently-labeled circulating cells” by Boutrus et al. published in J. Biomed. Opt., vol. 12, pg. 020507 (2007), which are herein incorporated by reference in their entirety. These articles disclose in-vivo methods of monitoring circulating cancer cells in animals that were injected with fluorescently labeled human cancer cells expressing green fluorescent protein (GFP). For example, both articles describe irradiating circulating cancer cells expressing GFP to excite those cells and quantifying the cancer cells by detecting the fluorescent radiation emitted by the excited GFP-labeled cells.

Likewise, circulating indicator cells can be detected via fluorescently labeling the cells, e.g., in a live subject, exciting the labeled cells with radiation and detecting fluorescent radiation emitted by the excited cells. For example, fluorescent-labeled dyes capable of binding to indicator cells can be injected intravenously into a subject. While in the subject's vasculature, the dye can come in contact with the indicator cells and bind to them through specific interactions. By way of example, an article entitled “Visualization and in situ analysis of leukocyte trafficking in the ankle joint in a systemic murine model of rheumatoid arthritis” by Gal et al., published in Arthritis and Rheumatism, vol. 52, pg. 3269 (2005), discloses methods for injecting fluorescent molecules into mice for in-vivo labeling of leukocytes. The authors disclose methods for administration of fluorescent membrane permeable dyes, such as rhodamine 6G, and administration of fluorescence (phycoerythrin)-conjugated monoclonal antibodies specific for surface receptors on leukocytes. The authors describe visualizing rhodamine 6G labeled cells in-vivo after minor surgical extraction of intraarticular tissue via intravital video microscopy, while phycoerythrin-labeled cells were extracted in a blood sample and quantified via traditional flow cytometric methods. An article entitled “In vivo flow cytometer for real-time detection and quantification of circulating cells” by Novak et al., published in Optics Letters, vol. 29 No. 1, pg. 77 (2004) discloses real-time detection and quantification of circulating leukocytes via fluorescence labeling. By way of further examples, an article entitled “Portable two-color in vivo flow cytometer for real-time detection of fluorescently-labeled circulating cells” by Boutrus et al., published in JBO Letters, vol. 12(2), pg. 020507-1 (2007) discloses real-time detection of fluorescently labeled stem cells.

Circulating cancer cells can also be identified via their autofluorescence. As known to those having ordinary skill in the art, autofluorescence can refer to the intrinsic fluorescence emitted by a cell, substance or object of interest without the use of fluorochrome staining or dyes when excited by suitable wavelengths of radiation. Depending on the cellular content of autofluorescent molecules (internal and external components of the cell), specific cell types can autofluoresce at different wavelengths.

By way of example, articles of Mujat et al. entitled “Endogenous optical biomarkers of normal and human papillomavirus immortalized epithelial cells.” International Journal of Cancer, vol. 122, pg. 363-371 (2008), Pavlova et al. entitled “Understanding the Biological Basis of Autofluorescence Imaging for Oral Cancer Detection: High Resolution Fluorescence Microscopy in Viable Tissue.” Clin. Cancer Res., vol. 14, pg. 2396 (2008), and DaCosta et al. entitled “Autofluorescence characterization of isolated whole crypts and primary cultured epithelial cells from normal, hyperplastic, and adenomatous colonic mucosa” J. Clin. Path., vol. 58, pg. 766 (2005), which are herein incorporated by reference in their entirety, provide examples of cancer cell autofluorescent signatures that can be used for detection of cancer cells.

Applicants have discovered that circulating indicator cells (e.g., immature leukocytes) can also be detected through autofluorescent radiation. By way of example, radiation at a wavelength of about 633 nm can be used to excite autofluorescent molecules in the circulating indicator cells and autofluorescence emitted by the circulating indicator cells can be detected in a wavelength range of about 650 nm to about 690 nm. In some embodiments, different radiation wavelengths can be utilized to excite molecules in cancer cells and circulating indicator cells, e.g., while the cells are circulating through a live subject, so as to elicit autofluorescent radiation from those cells. By way of example, excitation radiation wavelengths less than about 605 nm can be used for eliciting autofluorescent radiation from cancer cells and excitation wavelengths greater than about 620 nm can be used for eliciting autofluorescent radiation from circulating indicator cells. For example, in some cases, radiation at a wavelength of about 633 nm can be utilized to excite molecules in circulating indicator cells to emit autofluorescent radiation in a wavelength range of about 650 nm to about 690 nm while radiation at a wavelength of about 488 nm can be used to excite molecules in certain cancer cells to emit autofluorescent radiation at a wavelength of less than about 605 nm.

In other embodiments, autofluorescence can be employed in in-vitro studies to determine the ratio of cancer cells to indicator cells in a blood sample extracted from a subject. For example, the above excitation wavelengths can be employed in such in-vitro studies to elicit autofluorescence from cancer cells and/or indicator cells.

In some embodiments, in-vivo flow cytometry (IVFC) can be utilized to detect cancer cells through fluorescent labeling and to detect indicator cells via their autofluorescent signatures in order to determine the predictive ratio. For example, fluorescent and autofluorescent peaks can be counted concurrently over a given time interval and the ratio of counts corresponding to cancer cells and the indicator cells can be calculated as the predictive ratio.

In another aspect of determining the predictive ratio, ex-vivo techniques can be applied to samples extracted from a subject. In some embodiments, the predictive ratio can be determined by extracting a sample from a subject by methods known to those skilled in the art to determine the volume density of circulating cancer cells and indicator cells. For example, the indicator cells can be identified and counted via markers for immaturity and/or autofluorescence in the range of about 650 nm to about 690 nm in response to excitation by a wavelength of about 633 nm. Examples of methods for sample extraction can include, but are not limited to: blood draws, removal of interstitial fluid (excess lymph fluid buildup), ascites fluid drain (abdominal cavity fluids) and bone marrow aspiration. Skilled artisans will also be familiar with proper handling techniques of the samples. Methods for sample collection, proper handling and processing techniques are discussed by Henry in Clinical Diagnosis and Management by Laboratory Methods, 20^{th} Ed., W.B. Saunders Co., Philadelphia, 2001, which is herein incorporated by reference in its entirety.

By way of example, a blood sample can be extracted from a subject and cancer cells, if any, and the indicator cells in that sample volume can be counted, e.g., by employing known methods of ex-vivo flow cytometry. For example, U.S. Pat. No. 5,995,645 to Soenksen et al., which is herein incorporated by reference, discloses a method for ex-vivo flow cytometry that can be utilized for detecting cancer cell populations. A person skilled in the art can utilize the method of Soenksen et al. to determine the number of cancer cells within a specific volume, thereby determining the volume density of cancer cells. U.S. Pat. No. 6,004,816, which is herein incorporated by reference, to Mizukami et al. discloses a method for detecting leukocytes. A person skilled in the art can utilize a method similar to Mizukami et al. to count the number of circulating indicator cells in a specific volume and then determine the volume density of such cells. By way of example, methods similar to those of Soenksen et al. and Mizukami et al. can be applied concurrently to a sample extracted from a subject having cancer cells and/or indicator cells bound to dye conjugated molecules, such as fluorochrome conjugated antibody, to determine the volume densities of those cells and the volume densities can be utilized then to calculate the predictive ratio.

In addition to fluorescent labeling of cells as a method for detection, autofluorescent signatures, as described for in-vivo techniques, can also be used ex-vivo. By way of a nonlimiting example, a sample from a subject can be obtained and autofluorescence can be used to quantify the volume densities of cancer cells and indicator cells (e.g., leukocytes) present in that sample. In addition to fluorescence detection, other ex-vivo methods can be used to determine the quantity of circulating cancer cells and circulating indicator cells, such as, colony forming assays, immunohistochemistry and histology to quantify the number of cells in a specific volume of a sample.

In some embodiments, a combination of ex-vivo and in-vivo techniques can be employed to arrive at the predictive ratio. For example, the volume density of circulating indicator cells can be obtained in-vivo based on detection of autofluorescence emitted by those cells while the volume density of the cancer cells can be obtained ex-vivo, e.g., by applying flow cytometry to an extracted sample.

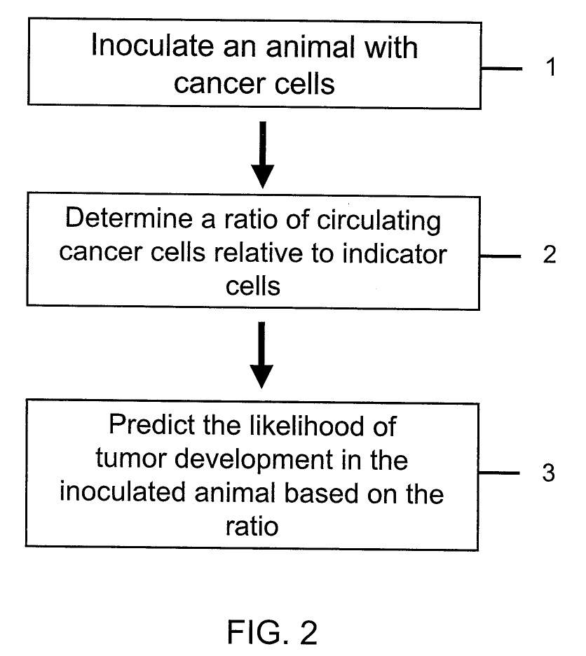

The methods of the invention for determining the likelihood of formation of a tumor and/or metastasis of a tumor can find a variety of applications, such as cancer diagnostic tools for animal studies and predictive tools for assessing progression of human cancers and/or response to therapy. By way of example, in one such application, the methods of the invention can be employed to obtain the likelihood that an animal inoculated with cancer cells would develop a tumor. For example, with reference to flow chart of FIG. 2, an animal can be inoculated with cancer cells (step 1), e.g., by injecting a quantity of cancer cells into the animal. Subsequently, a ratio of circulating cancer cells (i.e., cancer cells circulating in the animal's vasculature) relative to circulating indicator cells (e.g., circulating immature leukocytes) can be determined (step 2), e.g., by employing the techniques discussed above. The ratio can then be utilized to determine a likelihood that the animal would develop a tumor (step 3). As the ratio increases, the likelihood of tumor formation or progression assigned to the ratio also increases. For example, a ratio greater than about 0.26 can represent a likelihood of about 85% for tumor formation and a likelihood of about 43% for metastasis, while a ratio greater than about 0.5 could represent a likelihood of about 100% for tumor formation and metastasis.

Depending on the nature of the animal studies, a person of ordinary skill in the art can determine the most advantageous route for inoculation. Examples can include, but are not limited to, injecting the cancer cells intravenously, intraperitoneally, subcutaneously, intramuscularly, retro-orbitally, intradermally and intrathecally.

In some embodiments, the predictive ratio is determined through measurements of the cancer cells and indicator cells taken during a period in a range of about 1 day to about 14 days after the inoculation of the animal with cancer cells. In some other embodiments, the predictive ratio is determined through measurements taken at least 14 days, and in some instances more preferably 20 days, after inoculation of the animal. The type of cancer cells and the subject can influence the length of time after inoculation would yield the most accurate predictive ratio. Cancer cells with a long latency can be more accurately measured for predictive ratio in a time range of greater than 14 or even 20 days after inoculation. The subject can also influence the time after inoculation at which the measurements for predictive ratio can be taken. Large animals can typically require a longer time range after inoculation than smaller animals, such as rodents.

The ability to predict the incidence of cancer progression in research animal models can result in substantial savings in cost and time and can provide the potential for expanding research into new areas. The cost associated with animal disease models can be prohibitively high if the disease under study has a long latency prior to manifestation or a low frequency of occurrence. By determining the likelihood for tumor formation or progression in an animal model, researchers can predetermine which animals are likely to produce the most relevant data. In other words, researchers can reduce excessive costs associated with disease models having long latency and low frequency of occurrence.

In some cases, the inoculated animals that are not likely to develop a tumor in response to the inoculation, e.g., those exhibiting a likelihood of less than about 50%, or less than about 40%, can be discarded while retaining those that are likely to develop a tumor, e.g., those exhibiting a likelihood of greater than about 50%.

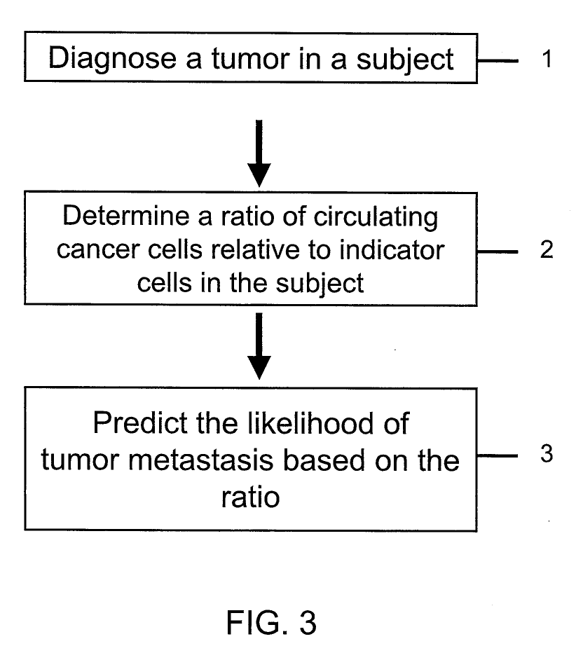

In addition to applicability of the method for cancer diagnostics in animal studies, the method of the invention can also be utilized as a predictive tool for human cancers. By way of example, in one such application, the methods of the invention can be employed to obtain the likelihood that a tumor would metastasize. For example, with reference to the flow chart of FIG. 3, a person is diagnosed with a tumor (step 1). Such diagnosis, including the type of cancer, can be done in a manner known in the art, e.g., by an oncologist or a pathologist. Subsequently, a ratio of circulating cancer cells (i.e., cancer cells circulating in the person's vasculature) relative to circulating indicator cells can be determined (step 2), e.g., by employing the techniques discussed above. The ratio can then be utilized to determine a likelihood that the tumor would metastasize (step 3).

Generally, as the ratio increases, the likelihood of tumor metastasis assigned to the ratio also increases. The specifics of the correlation between the ratio and the likelihood of tumor metastasis may vary from one cancer type to another, or between different animal species (e.g., mice and humans). However, the specifics of the correlation can be readily established for each case in accordance with the teachings of the invention without undue experimentation.

Once the likelihood of metastasis of a particular tumor has been determined, an appropriate therapy regimen based on that likelihood can be devised. For example, a more aggressive treatment regimen can be pursued for tumors that are more likely to metastasize (e.g., tumors for which the predictive ratio indicates a likelihood of metastasis greater than about 50% or greater than about 60% or greater than about 70%) to prevent metastasis while destroying the primary tumor. Alternatively, patients exhibiting a lower predictive ratio of cancer cells to indicator cells can receive less aggressive treatments with less serious side effects, which can be aimed at killing only the primary tumor.

Hence, in contrast to many conventional methods of cancer diagnosis and treatment that are either aimed at understanding and detecting metastases that have already occurred or rely on invasive procedures in predicting tumor progression (injection of radiolabelled dyes capable of identifying lymph nodes to be biopsied), many embodiments of the methods of the invention can predict cancer metastasis non-invasively.

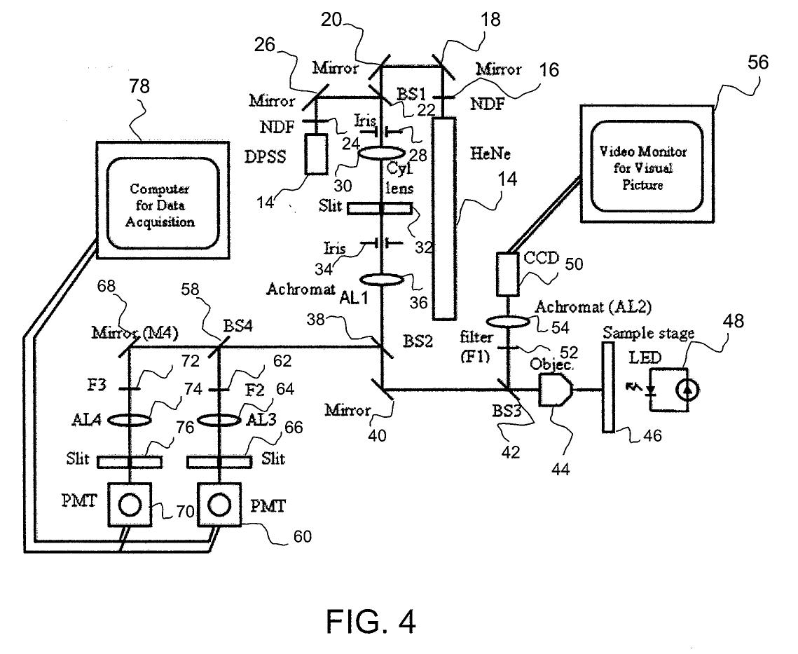

A variety of devices can be employed to carry out the methods of the invention. By way of example, FIG. 4 schematically depicts a two-color IVFC system 10 that can be employed in some embodiments to carry out the methods of the invention for counting circulating cancer cells labeled with green fluorescent protein (GFP-labeled cancer cells) as well as indicator cells that exhibit autofluorescence via detection of their autofluorescence. The exemplary system 10 includes two radiation sources 12 and 14, where the source 12 is a HeNe laser generating radiation at a wavelength of 633 nm and the source 14 is a diode-pumped solid state (DPSS) laser generating radiation at a wavelength of 488 nm. The radiation from the HeNe laser passes through a neutral density filter 16 to be reflected by mirrors 18 and 20 onto a beam splitter 22. The radiation from the DPPS in turn passes through a neutral density filter 24 to be reflected by a mirror 26 to the beam splitter 22. The radiation beam from the HeNe passes through the beam splitter 22 while the radiation beam from the DPPS is reflected by the beam splitter 22 so that the two beams propagate along a common path through an iris 28 to reach a cylindrical lens 30, which causes elongation of the beam's cross section in one direction.

The radiation beams are directed via the cylindrical lens 30 onto a slit 32 and an iris 34 to reach an achromat 36, which converges the radiation beams onto a beam splitter 38. The radiation beams pass through the beam splitter 38 and are reflected by a mirror 40 towards another beam splitter 42 to impinge on an objective 44, which focuses the beams onto a sample 46 under study.

To visualize the sample and align a portion of the sample, e.g., a blood vessel of the sample, with the incident radiation beams, a light emitting diode (LED) 48 transluminates the sample with the radiation passing through the sample collected via the objective lens to be directed by the beam splitter 42 to a CCD camera 50. More specifically, the radiation is directed through a filter 52 and is focused by a lens 54 onto the CCD camera 50. The output of the CCD camera is displayed on a visual monitor 56. Alternatively, a confocal imaging set-up could be employed to visualize the blood vessels in epi-illumination mode.

The fluorescence radiation emitted by the sample in response to the incident excitation beams is collected by the objective lens 44 and is transmitted, via passage through the beam splitter 42 and reflection from the mirror 40 and the beam splitter 38, to a beam splitter 58. A portion of the fluorescent radiation is reflected by the beam splitter 58 towards a photomultiplier tube (PMT) 60. A bandpass filter 62 placed in front of the PMT 60 allows transmission of fluorescence wavelengths in a range of about 510 nm to about 590 nm corresponding to fluorescent radiation emitted by the excited GFP-labeled cells. After passage of the fluorescent radiation through the filter, the radiation is focused by a lens 64 through a slit 66 onto the PMT 60.

Another portion of the returning fluorescent radiation passes through the beam splitter 58 and is reflected by the mirror 68 toward another PMT 70. A bandpass filter 72 placed in front of the PMT 70 allows the passage of the fluorescence wavelengths in a range of about 650 nm and 690 nm, corresponding to autofluorescent radiation generated by the sample. After passage through the filter 72, the fluorescent radiation is focused via a lens 74 through a slit 76 onto the PMT 70.

The signals generated by the PMTs 60 and 70 are transmitted to a data acquisition and analysis unit 78, e.g., a computer on which software for data acquisition and analysis is run. The analysis unit can be programmed to count the fluorescence peaks detected by the PMTs. In some cases, only those peaks having heights greater than a predefined threshold are counted and others are discarded as artifacts. Various methods of analyzing fluorescence data to count the fluorescence peaks known in the art can be utilized, such as those disclosed in U.S. Pat. No. 7,264,794 entitled “Methods Of In-Vivo Flow Cytometry,” which is herein incorporated by reference. Further, the analysis unit can calculate a ratio of the number of fluorescence peaks detected in the channel corresponding to labeled cancer cells to the number of fluorescence peaks detected, e.g., concurrently during the same time interval, corresponding to autofluorescent cells.

As noted above, the ratio can be employed to derive a likelihood of tumor formation and/or metastasis. In some cases, the correspondence between various values of the ratio and the likelihood of tumor formation and/or metastasis can be stored on the analysis module and accessed to correlate an experimentally obtained ratio to a probability value.

The teaching of a thesis entitled “Assessment of the Role of Circulating Breast Cancer Cells in Tumor Formation and Metastatic Potential Using In-Vivo Flow Cytometry,” by Derrick Hwu, which was presented in 2008 to Tufts University in partial fulfillment of the degree of Master of Science is herein incorporated by reference in its entirety.

### EXAMPLES

This invention is further illustrated by the following examples, which should not be construed as limiting. The following experiments are presented to further demonstrate various aspects of the invention, and are not necessarily intended to indicate optimal ways of carrying out the methods of the invention or optimal results that can be obtained by practicing the methods of the invention. Rather, the examples are presented for illustrative purposes, and should not be construed as limiting.

### Example 1

In one set of experiments, human breast cancer SUM1315 and DU4475 cells were transfected with GFP using the pBabe GFP-puro retrovirus and were cultured using a standard protocol. SUM1315 cell line is known to metastasize to the lungs and bones of NOD/SCID mice 30% and 20% of the time, respectively, while the DU4475 cells are known to metastasize to the lungs and brain 11% and 44% of the time, respectively. RMF/EG fibroblast cells were cultured and were used as an implantation control to ensure that the surgical implantation procedures were not a factor in subsequent IVFC measurements. Prior to surgical implantation, the cells were suspended in a 4:1 volume mixture of culture media and Matrigel (BD Bioscience). One million cells were injected into the mammary fat pads of 8 to 12 week old female NOD/SCID mice. Five mice were injected with SUM1315 cells, six mice were injected with DU4475 cells, and four control mice were injected with RMF/EG cells. One control mouse was not injected with any cells.

Surgical implantations and IVFC measurements were performed under anesthesia with a 7:1 mixture of 100 mg/kg ketamine and 20 mg/kg xylazine. IVFC measurements were performed on each mouse for eight to eleven weeks post surgery or until a tumor grew to be approximately 2 cm³ in volume. For most mice, measurements were taken one to three times before surgery, immediately after surgery, and every 3-4 days post surgery. Approximately 10 μL of blood was sampled from each mouse per day. The average number of detected fluorescent cells per microliter of blood was recorded by concurrently exciting the cells with radiation having wavelengths of 488 nm and 633 nm and detecting emitted fluorescent radiation at wavelengths of 510-590 nm in one detection channel and fluorescence radiation with wavelengths of 650-690 nm in another detection channel.

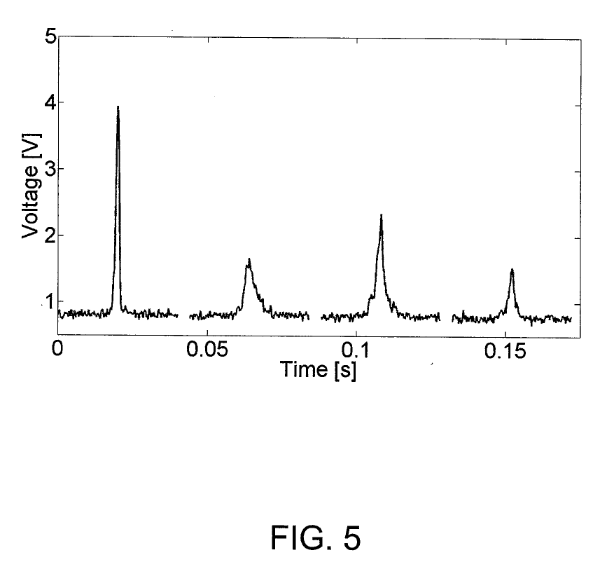

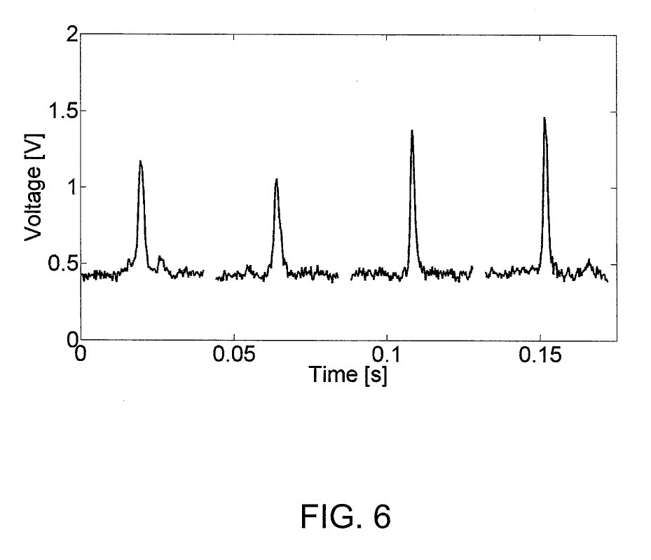

Representative peaks detected by green fluorescent protein (GFP) expressing cells or cancer cells (510-590 nm emission spectra when excited by 488 nm) are shown in FIG. 5. A significant variation was noted in the intensity of the detected peaks, consistent with previous IVFC studies, which are attributed to the intrinsic variations in the expression levels of GFP as well as the scattering and absorption properties of the sampled blood vessel and surrounding tissue. Red autofluorescent cells that exhibit autofluorescence (650-690 nm emission spectra collected in response to excitation by a radiation wavelength of 633 nm) were also detected in the arteries of each mouse, with some representative peaks detected as shown in FIG. 6. The mean height of the red peaks detected was approximately 0.202 volts with a standard deviation of 0.0995 volts. The mean height of the green peaks detected from the control mice was 0.265 volts with a standard deviation of 0.0738 volts while the mean height from the cancer mice was 0.275 volts with a standard deviation of 0.0845 volts.

Lung and brain tissues from implanted mice were fixed and stained with hematoxylin and eosin (H&E). Primary tumor sections (mammary fat pads) were also assessed histologically to assess stromal invasion by cancer cells. Mice with tumors that showed cancer cells growing and forming a tight border around the stroma indicated low to no potential for metastasis. In contrast, mice with tumors that clearly showed cancer cells invading through the border multiplying around the stroma cells indicated high potential for metastasis. One SUM1315 mouse was classified as positive for metastasis with fluorescence while two DU4475 mice were classified as positive with histology.

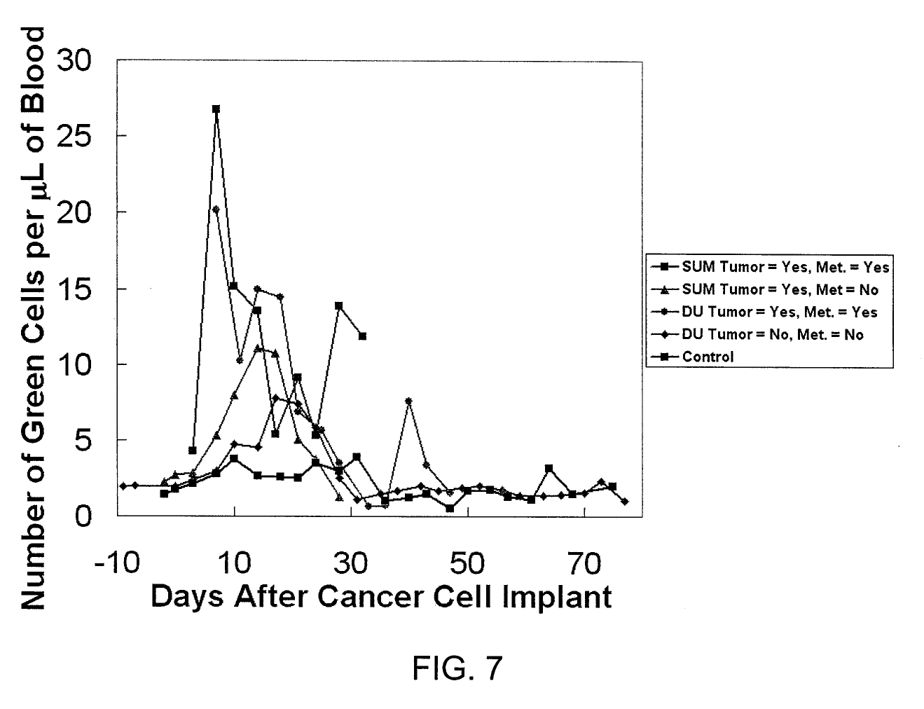

The average number of green fluorescent cells detected on each measurement day for each group of mice is shown in FIG. 7. Our data indicate that during the first few days post surgery, the average number of green fluorescent cells was significantly higher for mice that eventually developed tumors compared to those that did not. This suggests that successful tumor cell implantation can be predicted with IVFC within the first seven to ten days following implantation and in most cases several days to weeks before a palpable tumor can be observed.

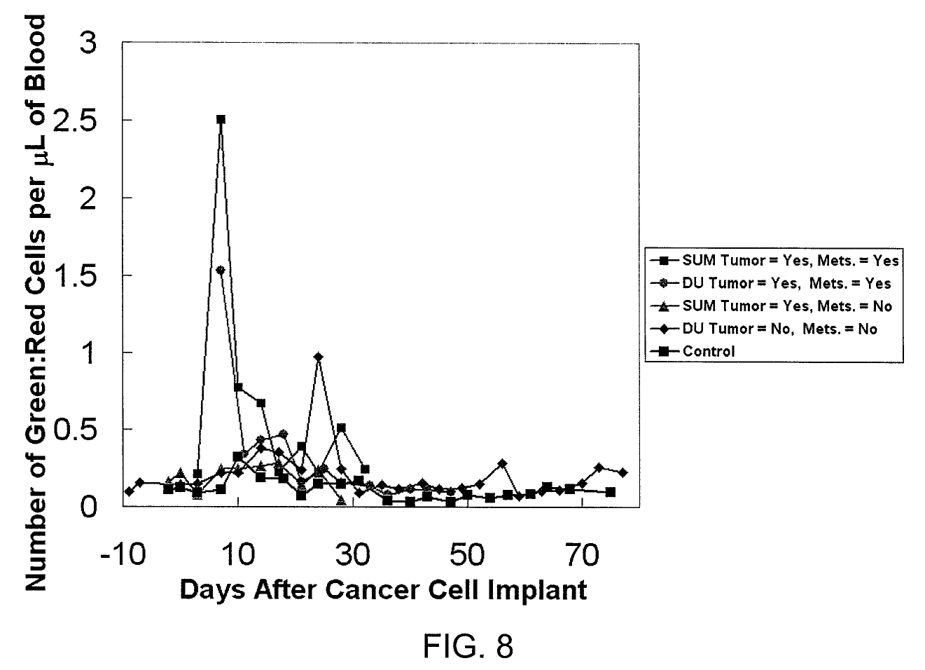

The average ratio of green to red fluorescent cells for each group of mice over the same period is shown in FIG. 8. We show the mean number of green autofluorescent cells from five groups of mice: control mice implanted with fibroblasts (n=4) or no cells (n=1) (squares, lower line); mice implanted with DU4475 cells that did not develop a primary or any metastatic tumors (n=4) (diamonds); mice implanted with DU4475 cells that did develop a primary tumor and a metastatic tumor (n=2) (circles); mice implanted with SUM1315 cells that developed a primary tumor but no metastasis (n=3) (triangles); and mice implanted with SUM1315 cells that developed a primary tumor and a metastatic tumor (squares, upper line). Even though the number of mice included in this study was small, this ratio was strikingly higher during the first two weeks following implantation for the mice that developed metastatic tumors than the mice that did not. Therefore, this ratio can be a predictive indicator of the metastatic potential of a tumor.

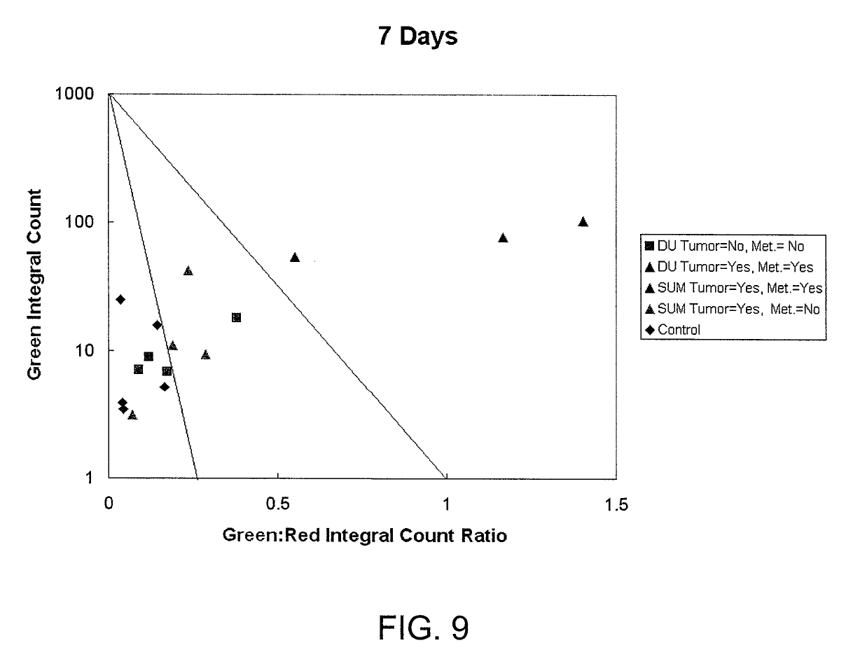

We considered both the maximum and the integrated number of green and red fluorescent peaks as indicators of tumor progression and metastasis over 7, 10, 14, 17 and 21 days of measurements following implantation. We found that the integrated number of green fluorescent peaks to the ratio of green to red fluorescent peaks within the first seven days following cell implantation offered the most accurate discrimination of the mice in groups: no tumors vs. primary tumor with no metastasis vs. primary tumor with metastasis. Using the simple lines of FIG. 9 drawn by visual inspection of the data, one could separate 3 out of 3 mice with metastasis, 6 out of 7 mice with tumors and 8 out of 9 mice with evidence of no tumors.

### Identification of Red Autofluorescent Cells

To determine the identity of the cells exhibiting red autofluorescence, a series of in-vitro antibody labeling experiments were performed. In one set of experiments, a total of 400 μL of blood was collected from two NOD/SCID mice with the submandibular bleeding technique. These mice had not undergone any procedure and therefore were not injected with any cancer cells or fibroblasts. The blood was lysed using a RBC lysis protocol and resuspended in DMEM. Using a FACS instrument, the green and red autofluorescent populations in the lysed blood were sorted and suspended in 100 μL of RPMI+2% FBS and placed under a fluorescent microscope. Trans-illumination, differential interface contrast, and fluorescent images were taken for green fluorescent cells. Since no appropriate filter combinations were available to detect red fluorescence, only trans-illumination and DIC images were taken for red autofluorescent cells.

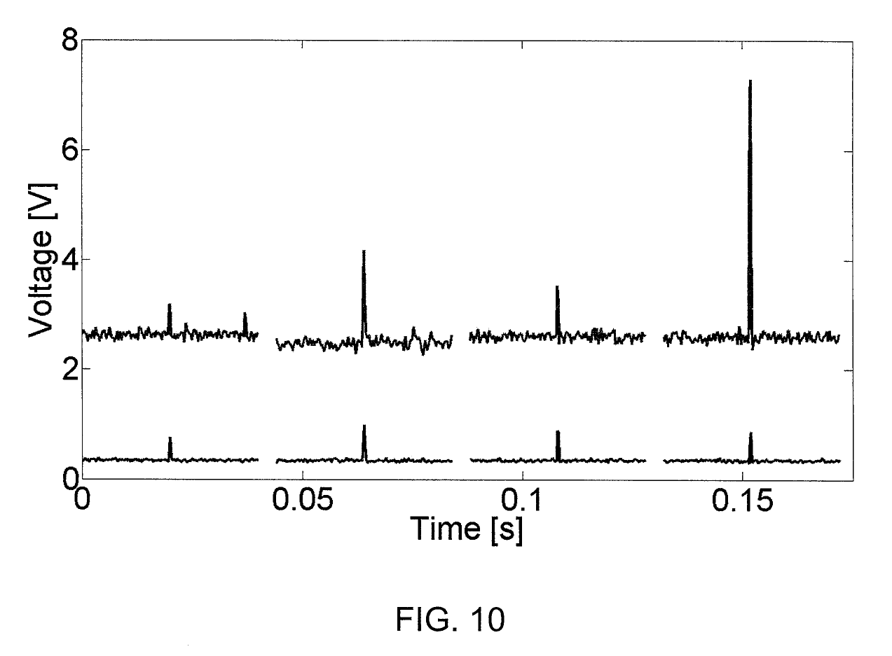

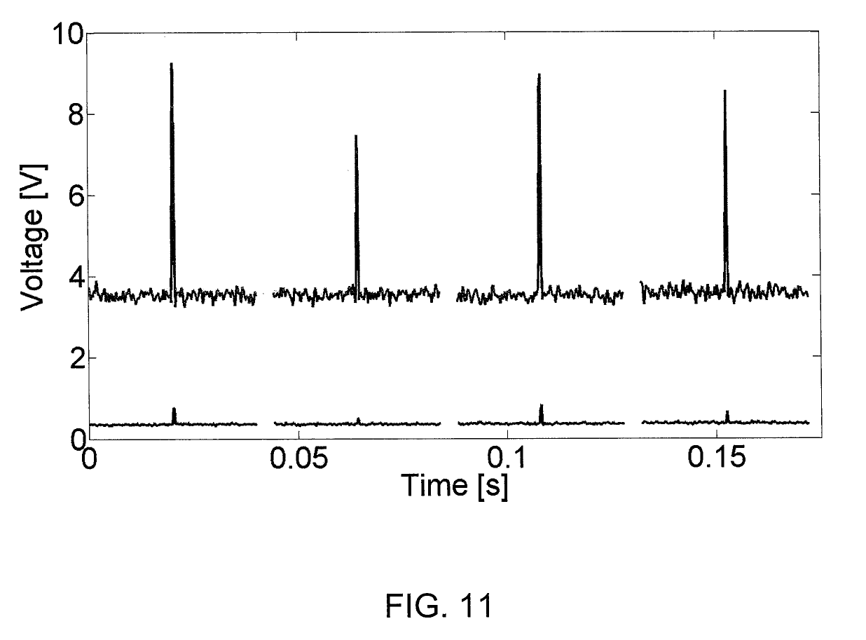

In another set of experiments, 220 μL of blood drawn from several NOD/SCID mice that had not undergone any procedure (1:3 volume mixture of whole blood to DMEM) was mixed with 5 μL of FITC anti-mouse CD31, or FITC anti-mouse Sca-1 antibody (220 μL of blood without any antibody served as a control). The mixtures were incubated at 4° C. in the dark for 30 minutes and flowed through 70 μm single channel polydimethylsiloxane (PDMS) microfluidic devices. Measurements were taken on the blood samples with the same IVFC instrument that was used for the animal studies. Examples of red autofluorescent peaks co-labeled with anti-CD31 FITC in FIG. 10 or anti-Sca-1 FITC antibody as shown in FIG. 11. Approximately 39% of the FITC-CD31 and 18% of the FITC Sca-1 peaks were correlated with the red autofluorescent cells. The combination of CD31 and Sca-1 are present on precursor endothelial cells and immature leukocytes as well as other hematopoietic cell populations such as natural killer (NK) cells, macrophages, and granulocytes. Based on this information and the number of detected cells, we hypothesize that the detected red autofluorescent cells comprise a subpopulation of immature circulating cells.

### Threshold for Cell Detection Via IVFC

The number of cells detectable immediately after inoculation depends on two parameters: 1. the number of cells inoculated and 2. an estimate of the number of circulating cells needed within an animal to be detectable by IVFC. We injected different doses of LNCaP cells, varying from 10³ to 10⁶, and performed IVFC measurements immediately following injection. These measurements illustrate that with only 1000 cells in the circulation, we can detect, unambiguously and reproducibly, a few cells in a 10-min recording period. The absolute number of detected cells per minute may vary from experiment to experiment, but could be the result of errors in estimating the cell concentration of the inoculum and variability in the number of cells successfully introduced in the mouse circulation. Small variations in the size of the selected arteries and in the flow velocity of cells can also affect the absolute number of detected cells. Nevertheless, a clear relationship is observed between the number of cells injected and the number of cells detected (cells/min). Potentially less than 1000 cells can be detected in the circulation by selecting larger arteries for measurements.

While the present invention has been described in terms of specific methods, structures, and devices it is understood that variations and modifications will occur to those skilled in the art upon consideration of the present invention. As well, the features illustrated or described in connection with one embodiment can be combined with the features of other embodiments. Such modifications and variations are intended to be included within the scope of the present invention. Those skilled in the art will appreciate, or be able to ascertain using no more than routine experimentation, further features and advantages of the invention based on the above-described embodiments. Accordingly, the invention is not to be limited by what has been particularly shown and described, except as indicated by the appended claims.

All publications and references are herein expressly incorporated by reference in their entirety. The terms “a” and “an” can be used interchangeably, and are equivalent to the phrase “one or more” as utilized in the present application. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed is:

1. A method for providing likelihood of occurrence of a cancerous tumor in an animal, comprising:
inoculating the animal with a plurality of cancer cells;
determining a ratio of a number of cancer cells relative to a number of circulating indicator cells in the inoculated animal's blood; and
correlating said ratio to a likelihood that the animal will develop at least one cancerous tumor.

2. The method of claim 1, wherein said step of determining the ratio comprises counting at least one of cancer cells or circulating indicator cells in-vivo in the animal's circulating blood.

3. The method of claim 1, wherein said step of determining the ratio comprises counting at least one of cancer cells or circulating indicator cells ex-vivo in at least one blood sample from the animal.

4. The method of claim 3, wherein the step of determining the ratio comprises:
drawing a volume of blood from the animal; and
counting cancer cells and circulating indicator cells in at least a portion of said blood volume.

5. The method of claim 1, further comprising determining said ratio during a time interval in a range of about 0 hours to about 14 days after the step of inoculating the animal.

6. The method of claim 1, further comprising determining said ratio during a time interval greater than 14 days after the step of inoculating the animal.

7. The method of claim 1, wherein the correlating step comprises assigning a probability for development of the tumor based on said measured ratio.

8. The method of claim 7, wherein said probability increases as the ratio increases.

9. The method of claim 1, wherein the correlating step comprises assigning a probability greater than about 85% for occurrence of the cancerous tumor if said ratio is greater than about 0.26.

10. The method of claim 1, wherein the circulating indicator cells exhibit makers for immaturity.

11. The method of claim 1, wherein the circulating indicator cells are immature leukocytes.

12. A method for providing likelihood of occurrence of a cancerous tumor in an animal, comprising:
inoculating the animal with a plurality of cancer cells expressing a fluorescent protein;
counting in-vivo the cancer cells in the animal's circulating blood by exciting said fluorescent protein and detecting fluorescent radiation emitted by said fluorescent protein in response to the excitation;
counting in-vivo circulating cells that emit autofluorescent radiation at a wavelength in a range of about 650 nm to about 690 nm in response to radiation with a wavelength of about 633 nm;
calculating a ratio of a count of the cancer cells relative to a count of the cells emitting autofluorescent radiation; and
determining a likelihood based on said ratio that at least one cancerous tumor will develop in the animal.

13. The method of claim 12, wherein said steps of counting the cancer cells and the cells emitting autofluorescence in a range of about 650 nm to about 690 nm are performed over a substantially equal time interval.

14. The method of claim 12, further comprising calculating said ratio during a time interval in a range of about 0 hours to about 14 days after the step of inoculating the animal.

15. The method of claim 14, further comprising determining said ratio after passage of at least 14 days from the step of inoculating the animal.

16. The method of claim 12, wherein the determining the likelihood step comprises assigning a probability for development of the tumor based on said measured ratio.

17. The method of claim 16, wherein said probability increases as the ratio increases.

18. The method of claim 12, wherein the determining the likelihood step comprises assigning a probability greater than about 85% for occurrence of the cancerous tumor if said ratio is greater than about 0.26.

19. The method of claim 12, wherein the cells emitting autofluorescence exhibit makers for immaturity.

20. The method of claim 12, wherein the cells emitting autofluorescence comprise immature leukocytes.

21. A method for determining metastatic potential of a tumor, comprising:
determining a ratio of a number of cancer cells to a number of circulating indicator cells in a patient's blood; and
correlating said ratio to a likelihood for metastasis of the tumor such that the lower said ratio the less likely for the tumor to metastasize.

22. The method of claim 21, wherein said step of determining the ratio comprises counting at least one of the cancer cells or circulating indicator cells in-vivo in the patient's circulating blood.

23. The method of claim 21, wherein the circulating indicator cells exhibit autofluorescence.

24. The method of claim 21, wherein said step of determining the ratio comprises counting at least one of the cancer cells or circulating indicator cells ex-vivo in at least one blood sample from the patient.

25. The method of claim 23, wherein the step of determining the ratio comprises:
drawing a volume of blood from the patient; and
counting cancer cells and indicator cells in at least a portion of said blood volume.

26. The method of claim 21, wherein the circulating indicator cells exhibit makers for immaturity.

27. The method of claim 21, wherein the circulating indicator cells comprise immature leukocytes

28. The method of claim 21, further comprising determining said ratio during a time interval in a range of about 0 hours to about 14 days after the step of inoculating the animal.

29. The method of claim 21, further comprising determining said ratio after passage of at least about 14 days from the step of inoculating the animal.

30. The method of claim 21, wherein the correlating step comprises assigning a probability for development of the tumor based on said measured ratio.

31. The method of claim 30, wherein said probability increases as the ratio increases.

32. The method of claim 21, wherein the correlating step comprises assigning a probability greater than about 85% for occurrence of the metastatic tumor if said ratio is greater than about 0.26.

33. A method for providing a likelihood of occurrence of a cancerous tumor in a patient, comprising:
counting in-vivo cancer cells in the patient's circulating blood that emit autofluorescent radiation at a wavelength less than about 605 nm in response to radiation at a wavelength of about 488 nm;
counting in-vivo circulating non-cancer cells that emit autofluorescent radiation at a wavelength in a range of about 650 nm to about 690 nm in response to radiation with a wavelength of about 633 nm;
calculating a ratio of the count of the cancer cells relative to the count of the circulating non-cancer cells; and
determining a likelihood based on said ratio that at least one metastatic tumor will develop in the patient.

34. The method of claim 33, wherein the step of determining the likelihood step comprises assigning a probability for development of the tumor based on said measured ratio.

35. The method of claim 34, wherein said probability increases as the ratio increases.

36. The method of claim 33, wherein the step of determining the likelihood comprises assigning a probability greater than about 43% for occurrence of the metastatic tumor if said ratio is greater than about 0.26.

37. The method of claim 33, wherein the step of determining the likelihood comprises assigning a probability of about 100% for occurrence of the metastatic tumor if said ratio is greater than about 0.5.

38. The method of claim 33, wherein the circulating non-cancer cells exhibit makers for immaturity.

39. The method of claim 33, wherein the circulating non-cancer cells comprise immature leukocytes.
